(19) United States
(12) Patent Application Publication
(10) Pub. No.: US 2006/0216242 A1
(43) Pub. Date: Sep. 28, 2006

(54) Suspending vehicles and pharmaceutical suspensions for drug dosage forms

(76) Inventors: Catherine Manya Rohloff, Los Altos, CA (US); Stephen Andrew Berry, Longview, WA (US); Eric William Weeks, Hayward, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/342,445

(22) Filed: Jan. 30, 2006

Related U.S. Application Data
(60) Provisional application No. 60/650,454, filed on Feb. 3, 2005.

(51) Int. Cl. A61K 48/00; A61K 38/28; A61K 38/31; A61K 38/30; A61K 38/21; A61K 38/20; A61K 38/19; A61K 38/18; A61K 38/22; A61K 38/17; A61L 9/04

(52) U.S. Cl. 424/45; 424/85.1; 424/85.2; 424/85.5; 424/85.6; 424/85.7; 424/94.1; 514/12; 514/44; 514/3

(57) ABSTRACT

Suspending vehicles and pharmaceutical suspensions that include a biocompatible polymer that can be combined with a hydrophobic solvent and a hydrophilic solvent to provide vehicles and suspensions that are substantially free of stiff gels upon contact with an aqueous medium are provided. Vehicles and suspensions remain flowable out of a pump-driven dosage form over the life of the dosage form. Such vehicles and suspensions are also biocompatible, suitable for creating and maintaining drug suspensions, and capable of providing stable drug formulations.

### CROSS-REFERENCE TO RELATED APPLICATIONS

This claims the benefit of priority under 35 U.S.C. § 119(e) from provisional U.S. Application Ser. No. 60/650,454, filed on Feb. 3, 2005, which is incorporated herein by reference in its entirety.

### FIELD OF THE INVENTION

The present invention relates to suspending vehicles and pharmaceutical suspensions in drug delivery systems and drug dosage forms utilizing the same.

### BACKGROUND OF THE INVENTION

Ensuring stability of pharmaceutical agents within dosage forms that include suspensions is important, for example, for effective dosaging and/or shelf-stability. Pharmaceutical suspensions can be used, for example, in osmotic drug delivery devices and injection depot devices. Osmotically-driven, also referred to as pump-driven, devices include those described in U.S. Pat. Nos. 5,985,305; 6,113,938; 6,132,420, 6,156,331; 6,395,292, each of which is incorporated herein by reference.

One approach to providing a stable suspension of a pharmaceutical agent is to provide a dosage form containing a suspending vehicle whose viscosity is sufficiently high to slow the sedimentation rate of the pharmaceutical agent. Typically, suspending vehicles contain a high viscosity, biocompatible polymer and a water-immiscible solvent. Water-immiscible solvents are typically chosen for their tendency to limit water ingress into drug dosage forms that are exposed to aqueous media, for example, bodily fluids. Such solvents have been shown to provide stable environments for pharmaceutically active agents such as proteins and peptides.

Some desirable polymers, such as polyvinyl pyrolidone (PVP), exhibit some amount of solubility in water. As such, phase behavior of some suspending vehicles at an organic/aqueous interface can be undesirable. In a region of dosage forms where a suspending vehicle is exposed to bodily fluids, limited quantities of water can reach the formulation due to the structure of exit ports out of the dosage forms. Phase separation can occur when polymer transfers from the suspending vehicle to an aqueous phase, and as a result of a limited availability of water, the aqueous phase may be highly concentrated in polymer. This can lead to highly viscous, almost solid formations. As such, under certain conditions, suspending vehicles comprising polymer in conjunction with a water-immiscible solvent may be difficult to pump through narrow exit ports of dosage forms. Further, reliability of dosage forms can be compromised by the formation of highly viscous, almost solid formations.

Hence, there exists a need to eliminate pluggage of discharge ports of implantable devices. Additionally, there is a need for suspending vehicles that are substantially resistant to phase separation, and for dosage forms which remain substantially homogenous for long periods of time.

### SUMMARY OF THE INVENTION

Generally, certain aspects of the invention provide suspending vehicles and pharmaceutical suspensions that include a biocompatible polymer that is combined with both a hydrophobic solvent and a hydrophilic solvent. Vehicles and suspensions remain flowable out of a pump-driven dosage form over the life of the dosage form. Such vehicles and suspensions are also biocompatible, suitable for creating and maintaining drug suspensions, and capable of providing stable drug formulations. Typically, vehicles and suspensions are substantially non-aqueous in order to limit ingress of water into a dosage form.

Through the use of a multi-component co-solvent in conjunction with a biocompatible polymer, suspending vehicles can be tailored to perform reliably in a wide range of dosage forms, for example, those that are pump-driven, under a variety of in vivo aqueous environments. In one aspect, the present invention provides a suspending vehicle in a pump-driven dosage form comprising a hydrophobic solvent, a hydrophilic solvent, and a biocompatible polymer, wherein the suspending vehicle is substantially free of stiff gels upon contact with an aqueous medium. In a preferred embodiment, the polymer comprises polyvinyl pyrolidone (PVP).

In some embodiments, a weight ratio of the hydrophobic solvent to the hydrophilic solvent is from about 0.1:99.9 to about 99.9:0.1; preferably, the weight ratio is from about 25:75 to about 99.9:0.1; more preferably, the weight ratio is from about 50:50 to about 99:1; and even more preferably, the weight ratio is from about 50:50 to about 90:10.

Examples in accordance with the present invention include vehicles having a weight ratio of a combination of the hydrophilic solvent and the hydrophobic solvent to the polymer of from about 20:80 to about 70:30; preferably, the weight ratio is from about 30:70 to about 70:30; and more preferably the weight ratio is from about 40:60 to about 55:45.

Hydrophobic solvents generally include, but are not limited to a carboxylic acid ester, a polyhydric alcohol, a polymer of a polyhydric alcohol, a fatty acid, an oil, propylene carbonate, an ester of a polyhydric alcohol, a triethylglyceride, or combinations thereof. In a detailed embodiment, the hydrophobic solvent comprises benzyl benzoate (BB), lauryl alcohol (LA), decyl alcohol, lauryl lactate (LL), myristyl lactate, myristyl alcohol, decyl lactate, Ceraphyl® 31, ethyl oleate, ethyl hexyl lactate, a vegetable oil, vitamin E, oleic acid, a mineral oil, or combinations thereof.

Hydrophilic solvents include, but are not limited to benzyl alcohol (BA), triacetin, diacetin, tributyrin, triethyl citrate, tributyl citrate, acetyl triethyl citrate, acetyl tributyl citrate, triethyl phosphate, diethyl phthalate, diethyl tartrate, polybutene, silicone fluid, glycerine, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol (GF), methyl acetate, ethyl acetate, methyl ethyl ketone, dimethylformamide, dimethyl sulfoxide, tetrahydrofuran, caprolactam, decylmethylsulfoxide, 1-dodecylazacyclo-heptan-2-one, polysorbate 80, tetraglycol, or combinations thereof.

Preferably, the suspending vehicle has a viscosity of from about 500 poise to about 70,000 poise at 37° C. and even more preferably, the viscosity is from about 5,000 poise to about 25,000 poise at 37° C.

Pharmaceutical suspensions are also provided by the present invention, the suspensions comprising a pharmaceutically active agent and a suspending vehicle, wherein the pharmaceutically active agent is suspended or dispersed in the suspending vehicle, wherein the suspending vehicle comprises a hydrophobic solvent, a hydrophilic solvent, and a biocompatible polymer and the suspending vehicle is substantially free of stiff gels upon contact with an aqueous medium. Preferably the pharmaceutical suspensions are substantially homogeneous for at least 3 months at 37° C.; more preferably for at least 6 months; and even more preferably for at least one year.

In a detailed embodiment, suspensions comprise pharmaceutically active agents including, but not limited to, ω-interferon, α-interferon, ⊕-interferon, γ-interferon, erythropoietin, granulocyte macrophage colony stimulating factor (GM-CSF), human growth hormone releasing hormone (huGHRH), insulin, desmopressin, infliximab, an antibody, an agent conjugated to a targeting ligand, bone morphogenic proteins, adrenocorticotropic hormone, angiotensin I, angiotensin II, atrial natriuretic peptide, bombesin, bradykinin, calcitonin, cerebellin, dynorphin N, alpha endorphin, beta endorphin, endothelin, enkephalin, epidermal growth factor, fertirelin, follicular gonadotropin releasing peptide, galanin, glucagon, glucagon-like peptide-1 (GLP-1), gonadorelin, gonadotropin, goserelin, growth hormone releasing peptide, histrelin, human growth hormone, insulin, leuprolide, LHRH, motilin, nafarerlin, neurotensin, oxytocin, relaxin, somatostatin, substance P, tumor necrosis factor, triptorelin, vasopressin, nerve growth factor, blood clotting factors, ribozymes, antisense oligonucleotide, or combinations thereof. Other desirable pharmaceutically active agents include, but are not limited to, risperidone, paliperidone, or combinations thereof.

Dosage forms of the present invention comprise a first wall that maintains its physical and chemical integrity during the life of the dosage form and is substantially impermeable to a pharmaceutical suspension; a second wall that is partially permeable to an exterior fluid; a compartment defined by the first wall and the second wall; a pharmaceutical suspension that is positioned within the compartment and comprises a hydrophobic solvent, a hydrophilic solvent, a biocompatible polymer, and a pharmaceutically active agent, wherein the suspending vehicle is substantially free of stiff gels upon contact with an aqueous medium; a pump in communication with the first wall, the second wall, and the compartment; and an exit port in communication with the compartment. In some embodiments, the pump comprises an osmotic pump.

It is preferable that upon contact with an aqueous medium, the pharmaceutical suspension is flowable through the exit port under a force exerted by the pump under normal operating conditions.

Kits in accordance with the present invention comprise a suspending vehicle and instructions for suspending or dispersing a pharmaceutically active agent therein to create a pharmaceutical suspension. Other kits further comprise a pump-driven dosage form and instructions for loading the dosage form with the pharmaceutical suspension. Still other kits comprise a dosage form and instructions for administering the dosage form.

Methods of the present invention include comprising administering the dosage forms to a mammal. Other methods comprise identifying a hydrophobic solvent; identifying a hydrophilic solvent; identifying a biocompatible polymer; and mixing the hydrophilic solvent, the hydrophobic solvent, and the biocompatible polymer to create a suspending vehicle; wherein the suspending vehicle is substantially free of stiff gels upon contact with an aqueous medium. Some methods further comprise adding a pharmaceutically active agent to the suspending vehicle to create a pharmaceutical suspension. Yet other methods further comprise adding the pharmaceutical suspension to a dosage form.

### BRIEF DESCRIPTION OF THE DRAWINGS

FIG. 1 shows viscosity of a BB/BA/PVP vehicle versus shear rate.

FIG. 2 depicts % by weight of ω-interferon detected in the protein particles that appears in the main protein peak and how this property changes over time. The particles containing the drug are suspended in BB/BA/PVP vehicles.

FIG. 3 depicts % oxidized ω-interferon by weight relative to the total ω-interferon detected in the protein particles suspended in BB/BA/PVP vehicles over time.

FIG. 4 depicts % deamidated co-interferon by weight relative to the total ω-interferon detected in the protein particles suspended in BB/BA/PVP vehicles over time.

FIG. 5 depicts % by weight of the total protein detected that is protein related to ω-interferon in the protein particles suspended in BB/BA/PVP vehicles over time.

FIG. 6 depicts % by weight of the total protein detected that is dimerized ω-interferon in the protein particles suspended in BB/BA/PVP vehicles over time.

FIGS. 7 and 8 show release of co-interferon over time from various dosage forms containing a pharmaceutical suspension including ω-interferon/BB/BA/PVP.

FIG. 9 shows viscosity of a GF/LL/PVP suspending vehicle versus shear rate.

FIG. 10 shows viscosity of a GF/LA/PVP suspending vehicle versus shear rate.

FIG. 11 shows a ternary phase diagram of vehicles containing polymer along with GF and/or LL or LA in the presence of PBS.

### DETAILED DESCRIPTION OF ILLUSTRATIVE EMBODIMENTS

Through the use of a multi-component co-solvent in conjunction with a biocompatible polymer, suspending vehicles can be tailored to perform reliably in a wide range of dosage forms, for example, those that are pump-driven, under a variety of in vivo aqueous environments, for a variety of pharmaceuticals. By reference to a multi-component solvent, it is meant that solvents are used that each provide different functionality. For example, a multi-component co-solvent can use a combination of at least one hydrophobic solvent and at least one hydrophilic solvent. Pharmaceutical suspensions using a multi-component co-solvent can provide flexibility in the preparation of formulations by improving, relative to suspensions that do not utilize a multi-component solvent, for example, the ability to adjust the solubility of the desired polymer, to titrate the formulation to improve protein stability, to affect the phase behavior in the presence of water, to adjust the phase transition temperature, and to reduce variability during manufacturing.

As such, using combinations of hydrophilic solvents and hydrophobic solvents in suspending vehicles helps to reducing plugging of dosage form outlets while also providing stable environments for desired drugs. With regard to vehicles having only a hydrophilic solvent in combination with a polymer, although such vehicles are usually miscible with water and do not lead to highly viscous polymer phases upon contact with water, the vehicles are limited in their ability to provide stable environment to pharmaceutically active agents such as proteins and peptides which are susceptible to degradation upon contact with water. On the other hand, vehicles utilizing only hydrophobic solvents, which are desirable for providing stable environments for proteins and peptides, tend to lead to phase separation and plugging upon contact with water. As such, adding an amount of hydrophilic solvent to a hydrophobic solvent-based vehicle generally leads to shifting the phase behavior of the vehicle upon contact with water. The use of a hydrophilic solvent leads to a softer aqueous phase then when only the hydrophobic solvent us present. Using a co-solvent does not always lead to complete miscibility between the vehicle and water, that is, there still may be two phases present once the vehicle is mixed with water, but nonetheless, the vehicle can still be effective in providing an environment for drug delivery.

Certain aspects of the invention provide suspending vehicles and pharmaceutical suspensions that may include a biocompatible polymer that can be combined with a hydrophobic solvent and a hydrophillic solvent to provide vehicles and suspensions that are substantially free of stiff gels upon contact with an aqueous medium. Vehicles and suspensions remain flowable out of a pump-driven dosage form over the life of the dosage form. Such vehicles and suspensions are also biocompatible, suitable for creating and maintaining drug suspensions, and capable of providing stable drug formulations. Generally, vehicles and suspensions are substantially nonaqueous in order to limit ingress of water into a dosage form.

Reference to “suspending vehicle” means that the pharmaceutically active agent is substantially insoluble therein. Materials that are substantially insoluble generally remain substantially in their original physical form throughout the lifespan of a dosage form containing the suspension. For example, solid particulates would generally remain particles. If necessary, the suspending vehicle may have other materials dissolved in it.

Reference to “flowable” means that the suspending vehicles and pharmaceutical suspensions are able to flow out of a dosage form despite the possible presence of a second phase. As such, although some polymer-based gels may be present in the vehicles and suspensions upon contact with an aqueous medium, the vehicles and suspensions are substantially free of stiff gels, that is free of gels that are hard enough to impede flow out of the dosage form. Hence, although gels may be present, they are sufficiently pliable to permit the vehicle or suspension to flow out of the dosage form, for example, an osmotic dosage form. Preferably, suspending vehicles and pharmaceutical suspensions according to the present invention remain flowable upon contact with an aqueous medium under normal operating conditions of the dosage form.

Reference to “hydrophilic solvent” means that the solvent in combination with a biocompatible polymer creates a mixture that is substantially miscible with water. As it is used herein, “miscible in water” refers to a mixture that, at a temperature range representative of a chosen operational environment, can be mixed with water at all proportions without resulting in a phase separation of the polymer from the solvent such that a highly viscous polymer phase is formed. A “highly viscous polymer phase” refers to a polymer-containing composition that exhibits a viscosity that is greater than the viscosity of the mixture of solvent and polymer, or of the suspending vehicle, before the mixture or vehicle is mixed with water. Benzyl alcohol (BA) is one example of a solvent that alone generally exhibits low solubility in water, but a combination of benzyl alcohol with a biocompatible polymer, for example, PVP, results in a vehicle that is water miscible, and thus, BA is considered a hydrophilic solvent.

Reference to “hydrophobic solvent” means that the solvent in combination with a biocompatible polymer is substantially immiscible with water. As it is used herein, “immiscible in water” refers to a mixture that, at a temperature range representative of a chosen operational environment, upon being mixed with water, there is at least one proportion of mixture to water that generally results in a phase separation of the polymer from the solvent such that a highly viscous polymer phase is formed. Reference to “substantially immiscible with water” means that some miscibility of the mixture with water is not precluded.

Preferably, the hydrophobic solvent and the hydrophilic solvent are miscible with each other. In some embodiments, a weight ratio of the hydrophobic solvent to the hydrophilic solvent is from about 0.1:99.9 to about 99.9:0.1; preferably, the weight ratio is from about 25:75 to about 99.9:0.1; more preferably, the weight ratio is from about 50:50 to about 99:1; and even more preferably, the weight ratio is from about 50:50 to about 90:10.

Examples in accordance with the present invention include vehicles having a weight ratio of a combination of the hydrophilic solvent and the hydrophobic solvent to the polymer being from about 20:80 to about 70:30; preferably, the weight ratio being from about 30:70 to about 70:30; and more preferably the weight ratio is from about 40:60 to about 55:45.

Hydrophobic Solvents

Many of the hydrophobic solvents useful in the invention are available commercially (Aldrich Chemicals, Sigma Chemicals) or may be prepared by conventional esterification of the respective arylalkanoic acids using acid halides, and optionally esterification catalysts, such as described in U.S. Pat. No. 5,556,905, and in the case of ketones, oxidation of their respective secondary alcohol precursors.

Solvents may be selected from aromatic alcohols, the lower alkyl and aralkyl esters of aryl acids such as benzoic acid, the phthalic acids, salicylic acid, lower alkyl (C₁ to C₆) esters of citric acid, such as triethyl citrate and tributyl citrate and the like, and aryl, aralkyl and lower alkyl ketones. Among preferred solvents are those having the following structural formulas (I), (II) and (III).

The aromatic alcohol has the structural formula (I)
Ar-(L)ₙ—OH (I)

wherein Ar is a substituted or unsubstituted aryl or heteroaryl group, n is zero or 1, and L is a linking moiety. Preferably, Ar is a monocyclic aryl or heteroaryl group, optionally substituted with one or more noninterfering substituents such as hydroxyl, alkoxy, thio, amino, halo, and the like. More preferably, Ar is an unsubstituted 5- or 6-membered aryl or heteroaryl group such as phenyl, cyclopentadienyl, pyridinyl, pyrimadinyl, pyrazinyl, pyrrolyl, pyrazolyl, imidazolyl, furanyl, thiophenyl, thiazolyl, isothiazolyl, or the like. The subscript “n” is zero or 1, meaning that the linking moiety L may or may not be present. Preferably, n is 1 and L is generally a lower alkylene linkage such as methylene or ethylene, wherein the linkage may include heteroatoms such as O, N or S. Most preferably, Ar is phenyl, n is 1, and L is methylene, such that the aromatic alcohol is benzyl alcohol.

The aromatic acid ester or ketone may be selected from the lower alkyl and aralkyl esters of aromatic acids, and aryl and aralkyl ketones. Generally, although not necessarily, the aromatic acid esters and ketones will respectively have the structural formula (II) or (III)

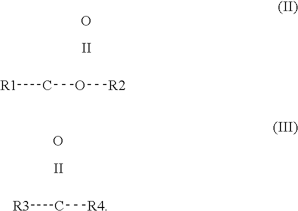

In the ester of formula (II), R1 is substituted or unsubstituted aryl, aralkyl, heteroaryl or heteroaralkyl, preferably substituted or unsubstituted aryl or heteroaryl, more preferably monocyclic or bicyclic aryl or heteroaryl optionally substituted with one or more non-interfering substituents such as hydroxyl, carboxyl, alkoxy, thio, amino, halo, and the like, still more preferably 5- or 6-membered aryl or heteroaryl such as phenyl, cyclopentadienyl, pyridinyl, pyrimadinyl, pyrazinyl, pyrrolyl, pyrazolyl, imidazolyl, furanyl, thiophenyl, thiazolyl, or isothiazolyl, and most preferably 5- or 6-membered aryl. R2 is hydrocarbyl or heteroatom-substituted hydrocarbyl, typically lower alkyl or substituted or unsubstituted aryl, aralkyl, heteroaryl or heteroaralkyl, preferably lower alkyl or substituted or unsubstituted aralkyl or heteroaralkyl, more preferably lower alkyl or monocyclic or bicyclic aralkyl or heteroaralkyl optionally substituted with one or more non-interfering substituents such as hydroxyl, carboxyl, alkoxy, thio, amino, halo, and the like, still more preferably lower alkyl or 5- or 6-membered aralkyl or heteroaralkyl, and most preferably lower alkyl or 5- or 6-membered aryl optionally substituted with one or more additional ester groups having the structure —O—(CO)—R1. Most preferred esters are benzoic acid and phthalic acid derivatives.

In the ketone of formula (III), R3 and R4 may be selected from any of the R1 and R2 groups identified above.

Art-recognized benzoic acid derivatives may be selected include, without limitation: 1,4-cyclohexane dimethanol dibenzoate, diethylene glycol dibenzoate, dipropylene glycol dibenzoate, polypropylene glycol dibenzoate, propylene glycol dibenzoate, diethylene glycol benzoate and dipropylene glycol benzoate blend, polyethylene glycol (200) dibenzoate, isodecyl benzoate, neopentyl glycol dibenzoate, glyceryl tribenzoate, pentaerylthritol tetrabenzoate, cumylphenyl benzoate, trimethyl pentanediol dibenzoate.

Art-recognized phthalic acid derivatives may be selected include: Alkyl benzyl phthalate, bis-cumyl-phenyl isophthalate, dibutoxyethyl phthalate, dimethyl phthalate, dimethyl phthalate, diethyl phthalate, dibutyl phthalate, diisobutyl phthalate, butyl octyl phthalate, diisoheptyl phthalate, butyl octyl phthalate, diisononyl phthalate, nonyl undecyl phthalate, dioctyl phthalate, di-isooctyl phthalate, dicapryl phthalate, mixed alcohol phthalate, di-(2-ethylhexyl)phthalate, linear heptyl, nonyl, phthalate, linear heptyl, nonyl, undecyl phthalate, linear nonyl phthalate, linear nonyl undecyl phthalate, linear dinonyl, didecyl phthalate (diisodecyl phthalate), diundecyl phthalate, ditridecyl phthalate, undecyldodecyl phthalate, decyltridecyl phthalate, blend (50/50) of dioctyl and didecyl phthalates, butyl benzyl phthalate, and dicyclohexyl phthalate.

Preferred solvents include aromatic alcohols, the lower alkyl and aralkyl esters of the aryl acids described above. Representative acids are benzoic acid and the phthalic acids, such as phthalic acid, isophthalic acid, and terephathalic acid. Derivatives of benzoic acid include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with benzyl benzoate being most especially preferred.

Other preferred solvents include carboxylic acid esters, polyhydric alcohols, polymers of polyhydric alcohols, fatty acids, oils, propylene carbonate, and esters of polyhydric alcohols. In other embodiments, hydrophobic solvents preferably include but are not limited to: benzyl benzoate, lauryl alcohol, decyl alcohol, lauryl lactate, myristyl lactate, myristyl alcohol, decyl lactate, Ceraphyl® 31, ethyl oleate, vegetable oils (for example, sesame, cottonseed, safflower, coconut, soybean, olive), vitamin E, or combinations thereof.

Hydrophilic Solvents

Hydrophilic solvents include but are not limited to: benzyl alcohol, triacetin, diacetin, tributyrin, triethyl citrate, tributyl citrate, acetyl triethyl citrate, acetyl tributyl citrate, triethyl phosphate, diethyl phthalate, diethyl tartrate, polybutene, silicone fluid, glycerine, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, 1-dodecylazacyclo-heptan-2-one, polysorbate 80, tetraglycol, or combinations thereof.

Polymers

Examples of polymers useful in forming a vehicle according to the present invention include, but are not limited to, polyesters such as PLA (polylactic acid) having an inherent viscosity in the range of about 0.5 to 2.0 i.v. and PLGA (polylacticpolyglycolic acid) having an inherent viscosity in the range of about 0.5 to 2.0 i.v., pyrrolidones such as polyvinylpyrrolidone (PVP) (having a molecular weight range of about 2,000 to 1,000,000), esters or ethers of unsaturated alcohols such as vinyl acetate, and polyoxyethylenepolyoxypropylene block copolymers such as Pluronic 105. If desired, more than one different polymer or grades of single polymer may be used to achieve a vehicle according to the present invention. PVP is a preferred polymer.

Pharmaceutically Active Agents

“Pharmaceutically active agent” refers to any biologically or pharmacologically active substance or antigen-comprising material; the term includes drug substances which have utility in the treatment or prevention of diseases or disorders affecting animals or humans, or in the regulation of any animal or human physiological condition and it also includes any biologically active compound or composition which, when administered in an effective amount, has an effect on living cells or organisms.

Pharmaceutical suspensions can be created by mixing the pharmaceutically active agent with the suspending vehicle. In some embodiments, the pharmaceutically active agent included in a suspension according to the present invention is generally degradable in water but generally stable as a dry powder at ambient and physiological temperatures. Active agents that may be incorporated into a suspension according to the invention include, but are not limited to, peptides, proteins, nucleotides, polymers of amino acids or nucleic acid residues, hormones, viruses, antibodies, etc. that are naturally derived, synthetically produced, or recombinantly produced. Preferably pharmaceutical suspensions remain substantially homogenous for about 3 months, even more preferably for about 6 months, and yet even more preferably, for about 1 year.

Preferably, the pharmaceutically active agent comprises ω-interferon, α-interferon, β-interferon, γ-interferon, erythropoietin, granulocyte macrophage colony stimulating factor (GM-CSF), human growth hormone releasing hormone (huGHRH), insulin, desmopressin, infliximab, antibody or an agent conjugated to a targeting ligand, risperidone, paliperidone, bone morphogenic proteins, adrenocorticotropic hormone, angiotensin I, angiotensin II, atrial natriuretic peptide, bombesin, bradykinin, calcitonin, cerebellin, dynorphin N, alpha endorphin, beta endorphin, endothelin, enkephalin, epidermal growth factor, fertirelin, follicular gonadotropin releasing peptide, galanin, glucagon, glucagon-like peptide-1 (GLP-1), gonadorelin, gonadotropin, goserelin, growth hormone releasing peptide, histrelin, human growth hormone, insulin, interferons, leuprolide, LHRH, motilin, nafarerlin, neurotensin, oxytocin, relaxin, somatostatin, substance P, tumor necrosis factor, triptorelin, vasopressin, growth hormone, nerve growth factor, blood clotting factors, ribozymes, antisense oligonucleotide, or combinations thereof.

Other desirable active substances to all aspects of the invention can be the so-called antiherpes virus agents which have been or are developed for the treatment of herpes virus infections [herpes simplex virus types 1 and 2 (HSV-1 and HSV-2)], varicella zoster virus (VZV), cytomegalovirus (CMV), Epstein-Barr virus (EBV)]. The antiherpes virus agents include antiviral drugs and prodrugs thereof, such as nucleosides, nucleoside analogues, phosphorylated nucleosides (nucleotides), nucleotide analogues and salts, complexes and prodrugs thereof, e.g., guanosine analogues, deoxyguanosine analogues, guanine, guanine analogues, thymidine analogues, uracil analogues and adenine analogues. Antiherpes virus agent for use either alone or in combination in a composition according to the present invention can be selected from acyclovir, famciclovir, deciclovir, penciclovir, zidovudin, ganciclovir, didanosin, zalcitabin, valaciclovir, sorivudine, lobucavir, brivudine, cidofovir, n-docosanol, ISIS-2922, and salts, prodrugs, derivatives, analogues, and combinations thereof.

Other drugs which in themselves have a low water solubility, or the salts, esters, prodrugs or precursors of which have a low solubility may also be desirable in the compositions of the invention. Furthermore, it may be desirable to combine some active ingredients. As such, any of the foregoing examples can either alone or in combination can be incorporated in a composition according to the present invention. For example, a combination of active ingredients may include an anti-herpes virus agent and a glucocorticosteroid.

With respect to pharmaceutically active agents, pharmaceutical suspensions located in pump-driven dosage forms preferably comprise from about 0.1% to about 15% pharmaceutically active agent by weight, and more preferably from about 0.2% to about 2%. Typically, the corresponding drug particle loadings are preferably approximately from about 3% to about 30% by weight of the suspensions, and more preferably from about 3% to about 13%.

Pharmaceutical suspensions according to the present invention may include any pharmaceutically active agent that either exhibits desired solubility characteristics or may be prepared as a particulate material exhibiting desired solubility characteristics. Agents may be provided in the form of pharmaceutically acceptable salts, including salts with inorganic acids, organic acids, inorganic bases, organic bases, or combinations thereof. In some aspects, the agents are a biomolecular material, such as a peptide or protein that has biological activity or that may be used to treat a disease or other pathological condition. Analogs, derivatives, antagonists, and agonists of the exemplary peptides and proteins described may also be used. Agents are not limited to a biomolecular material. The drug may be any compound or material, including any medicine, vitamin, nutrient, or food supplement, which is capable of providing a therapeutic or beneficial affect when administered to an environment of operation and can be prepared as a particulate material exhibiting desired solubility characteristics.

The active agents included in a suspension according to the present invention may also include lipoproteins and post translationally modified forms, e.g., glycosylated proteins, as well as proteins or protein substances which have D-amino acids, modified, derivatized or non-naturally occurring amino acids in the D- or L-configuration and/or peptomimetic units as part of their structure. Specific examples of materials that may be included in as the pharmaceutically active agent in a suspension of the present invention include, but are not limited to, baclofen, GDNF, neurotrophic factors, conatonkin G, Ziconotide, clonidine, axokine, anitsense oligonucleotides, adrenocorticotropic hormone, angiotensin I and II, atrial natriuretic peptide, bombesin, bradykinin, calcitonin, cerebellin, dynorphin N, alpha and beta endorphin, endothelin, enkephalin, epidermal growth factor, fertirelin, follicular gonadotropin releasing peptide, galanin, glucagon, gonadorelin, gonadotropin, goserelin, growth hormone releasing peptide, histrelin, insulin, interferons, leuprolide, LHRH, motilin, nafarerlin, neurotensin, oxytocin, relaxin, somatostatin, substance P, tumor necrosis factor, triptorelin, vasopressin, growth hormone, nerve growth factor, blood clotting factors, ribozymes, and antisense oligonucleotides. Analogs, derivatives, antagonists agonists and pharmaceutically acceptable salts of each of the above mentioned active agents may also be used in formulating an active agent suspension of the present invention. Preferably, the active agents provided in a suspension of the present invention exhibits little or no solubility in the chosen suspension vehicle.

The active agents can be in various forms, such as uncharged molecules, molecular complexes, pharmacologically acceptable acid or base addition salts such as hydrochlorides, hydrobromides, sulfate, laurylate, palmitate, phosphate, nitrate, borate, acetate, maleate, tartrate, oleate, and salicylate. For acidic drugs, salts of metals, amines or organic cations, for example quaternary ammonium can be used. Derivatives of drugs such as esters, ethers and amides can be used alone or mixed with other drugs. Also, a drug that is water insoluble can be used in a form that on its release from a device, is converted by enzymes, hydrolyzed by body pH or other metabolic processes to the original form, or to a biologically active form.

With respect to pharmaceutically acceptable excipients and other processing aids, it is preferable that the drug particles incorporate any such excipients and/or aids into the solid drug particulate to be delivered from a suspension dosage form. As such, reference to drug particles or pharmaceutically active agents, includes any such excipients or aids incorporated therein.

Other desirable pharmaceutically active agents include, but are not limited to, the following groups: sodium fluoride, anti-inflammatory drugs such as, e.g., ibuprofen, indomethacin, naproxen, diclofenac, tolfenamic acid, piroxicam, and the like; narcotic antagonists such as, e.g., naloxone, nalorphine, and the like; antiparkinsonism agents such as, e.g., bromocriptine, biperidin, benzhexol, benztropine, and the like; antidepressants such as, e.g., imipramine, nortriptyline, pritiptylene, and the like; antibiotic agents such as, e.g., clindamycin, erythromycin, fusidic acid, gentamicin, mupirocien, amfomycin, neomycin, metronidazole, silver sulphadiazine, sulphamethizole, bacitracin, framycetin, polymycin B, acitromycin, and the like; antifungal agents such as, e.g., miconazol, ketoconazole, clotrimazole, amphotericin B, nystatin, mepyramin, econazol, fluconazol, flucytocine, griseoftdvin, bifonazole, amorolfine, mycostatin, itraconazole, terbenafine, terconazole, tolnaftate, and the like; antimicrobial agents such as, e.g., metronidazole, tetracyclines, oxytetracycline, and the like; antiemetics such as, e.g., metoclopramide, droperidol, haloperidol, promethazine, and the like; antihistamines such as, e.g., chlorpheniramine, terfenadine, triprolidine, and the like; antimigraine agents such as, e.g., dihydroergotamine, ergotamine, pizotyhne, and the like; coronary, cerebral or peripheral vasodilators such as, e.g., nifedipine, diltiazem, and the like; antianginals such as, e.g., glyceryl nitrate, isosorbide denitrate, molsidomine, verapamil, and the like; calcium channel blockers such as, e.g., verapamil, nifedipine, diltiazem, nicardipine, and the like; hormonal agents such as, e.g., estradiol, estron, estriol, polyestradiol, polyestriol, dienestrol, diethylstilbestrol, progesterone, dihydroergosterone, cyproterone, danazol, testosterone, and the like; contraceptive agents such as, e.g., ethynyl estradiol, lynestrenol, etynodiol, norethisterone, mestranol, norgestrel, levonorgestrel, desogestrel, medroxyprogesterone, and the like; antithrombotic agents such as, e.g., heparin, warfarin, and the like; diuretics such as, e.g., hydrochlorothiazide, flunarizine, minoxidil, and the like; antihypertensive agents such as, e.g., propanolol, metoprolol, clonidine, pindolol, and the like; corticosteroids such as, e.g., beclomethasone, betamethasone, betamethasone-17-valerate, betamethasone-dipropionate, clobetasol, clobetasol-17-butyrate, clobetasol-propionate, desonide, desoxymethasone, dexamethasone, diflucortolone, flumethasone, flumethasone-pivalate, fluocinolone acetonide, fluocinonide, hydrocortisone, hydrocortisone-17-butyrate, hydrocortisone-buteprate, methylprednisolone, triamcinolone acetonide, budesonide, halcinonide, fluprednide acetate, alklometasone-dipropionate, fluocortolone, fluticason-propionate, mometasone-furate, desoxymethasone, diflurason-diacetate, halquinol, cliochinol, chlorchinaldol, fluocinolone-acetonid, and the like; dermatological agents such as, e.g., nitrofurantoin, dithranol, clioquinol, hydroxyquinoline, isotretionin, methoxsalen, methotrexate, tretionin, trioxsalen, salicylic acid, penicillamine, and the like; steroids such as, e.g., estradiol, progesterone, norethindrone, levonorgestrol, ethynodiol, levenorgestrel, norgestimate, gestanin, desogestrel, 3-keton-desogestrel, demegestone, promethoestrol, testosterone, spironolactone, and esters thereof, nitro compounds such as, e.g., amyl nitrates, nitroglycerine and isosorbide nitrates, opioid compounds such as, e.g., morphine and morphine-like drugs such as buprenorphine, oxymorphone, hydromorphone, levorphanol, fentanyl and fentanyl derivatives and analogues, prostaglandins such as, e.g., a member of the PGA, PGB, PGE, or PGF series such as, e.g., misoprostol, dinoproston, carboprost or enaprostil, a benzamide such as, e.g., metoclopramide, scopolamine, a peptide such as, e.g., growth hormone releasing factors, growth factors (epidermal growth factor (EGF), nerve growth factor (NGF), TGF, PDGF, insulin growth factor (IGF), fibroblast growth factor (FGFα, FGFβ, etc.), and the like), somatostatin, calcitonin, insulin, vasopressin, interferons, interleukins, e.g., IL-2, IL-12, IL-21, urokinase, serratiopeptidase, superoxide dismutase (SOD), thyrotropin releasing hormone (TRH), luteinizing hormone releasing hormone (LH-RH), corticotrophin releasing hormone (CRF), growth hormone releasing hormone (GHRH), oxytocin, erythropoietin (EPO), colony stimulating factor (CSF), and the like, a xanthine such as, e.g., caffeine, theophylline, a catecholamine such as, e.g., ephedrine, salbutamol, terbutaline, a dihydropyridine such as, e.g., nifedipine, a thiazide such as, e.g., hydrochlorotiazide, flunarizine, others such as, e.g., propanthelin, silver nitrate, enzymes like Streptokinases, Streptodases, vitamins like vitamin A, tretionin, isotretionin, acitretin, vitamin D, calcipotriol, interferon-α-2b, selen disulfide, pyrethione.

It will be understood that the compositions of the invention can also comprise combinations of active substances, e.g., an active substance together with a potentiator therefor. It will of course also be understood that in the aspects of the invention wherein there is no specific requirement to the active substance, e.g., with respect to solubility, any substance which has a therapeutic or prophylactic activity can be incorporated in the composition.

Dosage Forms

Suspending vehicles and pharmaceutical suspensions can be prepared for use in all types of dosage forms, e.g., oral suspensions, ophthalmologic suspensions, implant suspensions, injection suspensions, and infusion suspensions. A preferred dosage form is an implantable osmotic dosage form. Osmotically-driven, also referred to as pump-driven, devices include those described in U.S. Pat. Nos. 5,985,305; 6,113,938; 6,132,420, 6,156,331; 6,395,292, each of which is incorporated herein by reference.

It is preferable that the suspending vehicle, the hydrophilic solvent, and/or the hydrophobic solvent are physiologically acceptable for a desired route of administration, for example, there are no adverse biological responses by the recipient of the suspension upon administration. In some embodiments of the present invention, it is preferable that the components are suitable for parenteral routes of administration, including but not limited to injection, infusion, or implantation.

### EXAMPLES

Below are several examples of specific embodiments for carrying out the present invention. The examples are offered for illustrative purposes only, and are not intended to limit the scope of the present invention in any way.

### Example 1

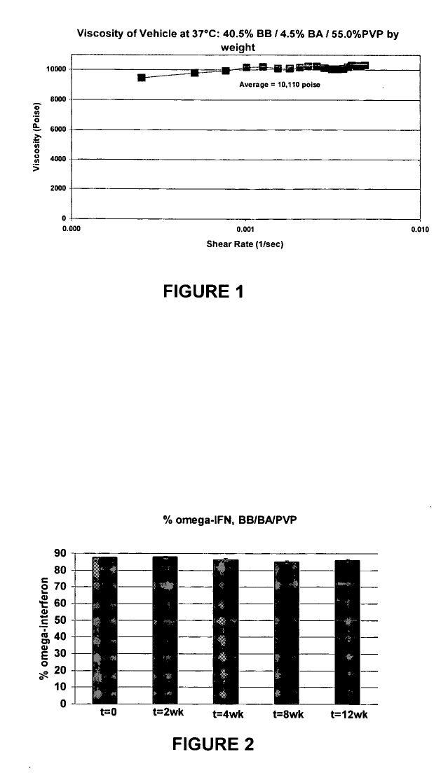

Benzyl benzoate (BB) was blended with benzyl alcohol (BA) as a co-solvent mixture for PVP to create a suspending vehicle. Appropriate amounts of benzyl alcohol and benzyl benzoate were mixed using a stir bar for 15-30 minutes at room temperature. PVP was then added to the solvent mixture and mixed under heat until PVP was dissolved. FIG. 1 demonstrates that viscosity is generally independent of shear rate. Average viscosity at 37° C. was 10,110 poise. Table 1 summarizes the phase behavior of the suspending vehicles, which were prepared at a solvent to polymer weight ratio of 50:50. Descriptions refer to, if any, the aqueous phases.

TABLE 1
Phase Behavior of BB/BA/PVP Vehicles

| | 10% PBS | 25% PBS | 50% PBS |
|---|---|---|---|
| BA:BB | (above baseline | (above baseline | (above baseline |
| By wt | moisture) | moisture) | moisture) |
| 10:90 | Two Phases: | Two Phases: | Two Phases: |
| | Semi-Firm | Liquid | Liquid |
| 24:76 | Two Phases: | Two Phases: | Two Phases: |
| | Soft | Liquid | Liquid |
| 50:50 | Two Phases: | Two Phases: | Two Phases: |
| | Liquid | Liquid | Liquid |
| 62.5:37.5 | One Phase | Two Phases | Two Phases: |
| | | | Liquid |
| 75:25 | One Phase | Two Phases | Two Phases |
| 85:15 | One Phase | Two Phases | Two Phases |
| 95:5 | One Phase | One Phase | Two Phases |
| 100:0 | One Phase | One Phase | Two Phases: |
| | | | Liquid |

### Example 2

Co-solvents BB and BA were combined in a weight ratio of 90:10. A suspending vehicle was prepared by mixing the co-solvent mixture and PVP in a weight ratio of 47.5:52.5. Viscosity of the suspending vehicle was 8,400 poise at 37° C. A spray-dried protein, ω-interferon, was added to the suspending vehicle at a total particle loading of 10% by weight, corresponding to 1.7% by weight co-interferon, to create a pharmaceutical suspension. The protein particles comprised 1:2:1:2.15 by weight ratio of co-interferon to sucrose to methionine to citrate.

### Example 3

In vitro stability testing was conducted on the pharmaceutical suspensions prepared in Example 2. The original co-interferon particle characteristics are listed in Table 2. There was minimal change in protein characteristics over time.

TABLE 2
Characteristics of ω-Interferon Used in BB/BA/PVP Suspensions

| | |
|---|---|
| Total % Oxid (confidence interval) | 1.71 (0.10) |
| % Deamid (confidence interval) | 1.5 (0.02) |
| Total % Related Protein (confidence interval) | 8.0 (0.07) |
| % Dimer | 0.00 |
| % ω-Interferon (confidence interval) | 88.8 (0.8) |

The suspensions were loaded into osmotic dosage forms: 3 dosage forms (N=3) containing the suspensions from Example 2 were used. A membrane end of the dosage form was placed in a container of Phosphate buffer (pH=7.4) and an exit port of the dosage form was placed in a Citrate buffer (pH=2.0). The buffer containers and dosage forms were placed in an incubator at 37° C. Over a period of 12 weeks, the buffer holding the exit port was analyzed weekly to analyze the quantity of protein released.

As shown in FIG. 2, which depicts % by weight of the main peak of total ω-interferon in the suspended protein particles over time, measured levels of ω-interferon did not change in the BB/BA/PVP vehicles. Levels of “pure” ω-interferon were measured via rp HPLC.

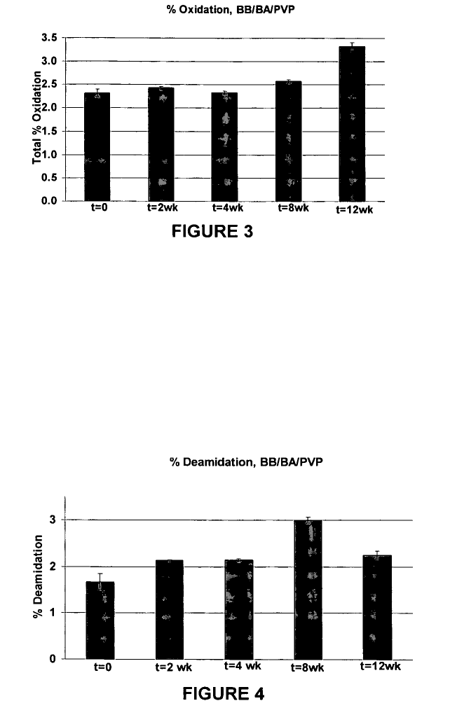

FIG. 3, which depicts % by weight of oxidized (o-interferon relative to the total ω-interferon detected in the suspended protein particles over time, demonstrates that protein oxidation is unchanged between 0 and 8 weeks, and may increase slightly after 12 weeks. In the BB/BA/PVP vehicles, the oxides measured 2.32 weight % at 0 weeks and 3.32 weight % at 12 weeks.

FIG. 4, which depicts % by weight of deamidated ω-interferon relative to the total ω-interferon detected in the suspended protein particles over time, indicates that protein deamidation is unchanged between 0 and 12 weeks.

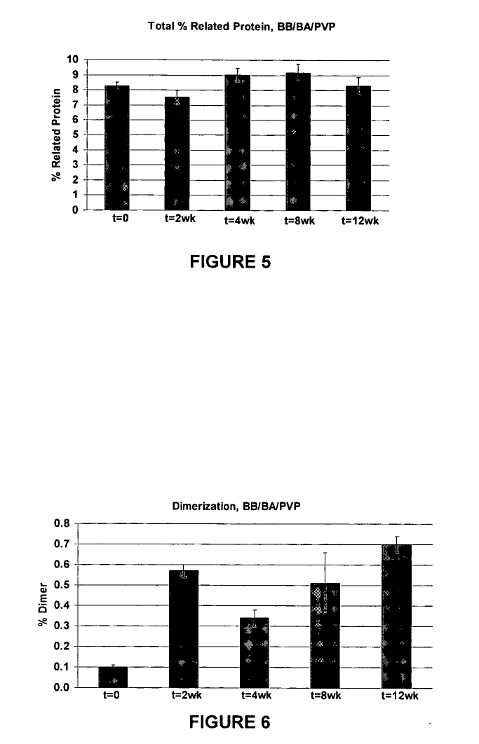

In FIG. 5, which depicts % by weight of related protein relative to the total ω-interferon detected in the suspended protein particles over time, there is no substantial change in levels of related protein from 0 to 12 weeks.

FIG. 6, which depicts % by weight of dimerized (o-interferon relative to the total c-interferon detected in the suspended protein particles over time, shows that the measured levels of dimers increased from 0 to 2 weeks and did not increase from 2 to 12 weeks. In the BB/BA/PVP vehicles, there was an increase from 0.01 weight % dimer at 0 weeks to 0.70 weight % dimer after 12 weeks.

### Example 4

Release rates of ω-interferon were analyzed using the pharmaceutical suspensions vehicles prepared in Example 2.

The suspensions were loaded into various types of osmotic dosage forms: primed spiral diffusion moderators (N=12), unprimed spiral diffusion moderators (N=12), primed capillary diffusion moderators (N=3), and unprimed capillary diffusion moderators (N=2). By “primed” it is meant that the membrane has been hydrated and formulation has begun emerging from the system into a dry vial before starting the test. Correspondingly, by “unprimed” it is meant that the membrane has not been hydrated and the system will begin pumping directly into the buffer. A membrane end of the dosage form was placed in a container of buffer and an exit port of the dosage form was placed in a different container containing 50 mM citrate buffer, pH of 6.0. Over a period of 89 days, the buffer holding the exit port was regularly analyzed for total protein, where total protein equaled the amount of soluble protein added to the amount of protein recovered from treatment with guanidine. None of the dosage forms failed during the 89 day trial period. That is, none of the dosage forms failed to deliver protein.

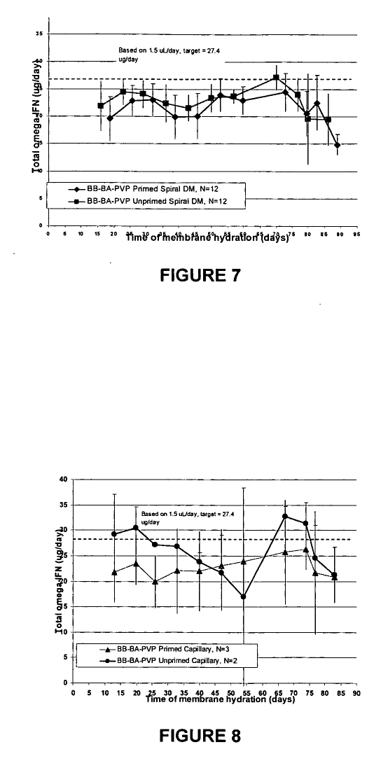

The dosage forms were set-up to nominally deliver 1.5 μL per day of suspension, which targeted delivery of 27.4 μL per day of the protein. FIG. 7 shows release of ω-interferon which was suspended in a BB/BA/PVP suspending vehicle through a spiral diffusion moderators over 89 days. During the test period, membranes of all of the dosage forms remained in tact and total protein release was near the target.

FIG. 8 shows release of ω-interferon which was suspended in a BB/BA/PVP suspending vehicle through a capillary tube over 89 days. During the test period, membranes of all of the dosage forms remained in tact and total protein release was near the target.

### Example 5

Glycofurol (GF) was combined with another solvent to alter properties of a suspending vehicle, for example, to soften a second phase formed when the vehicle is exposed to water. Typically, an aqueous phase contains some amount of polymer which has hardened upon contact with water. Solvent combinations were screened to evaluate phase behaviors of different solvent ratios. Properties of the co-solvent vehicle in the presence of PBS at two levels (10% and 20% by weight) were evaluated.

Co-solvent mixtures were prepared at weight ratios of glycofurol to lauryl lactate (LL) of 0:100, 10:90, 25:75, 50:50, and 75:25. For each ratio of GF to LL, the co-solvents were weighed and combined together in a jar. Dried polymer, PVP, was weighed and added to the jar at a solvent to polymer ratio of 45:55 for create a suspending vehicle. The suspending vehicle was mixed by hand with a spatula. After sufficient mixing, the jar was placed in an oven at 65° C. for approximately 1 hour to further dissolve any PVP. The jars were then placed in a vacuum oven and subjected to a vacuum for approximately 1 hour to remove any air entrapped in the suspending vehicle during mixing.

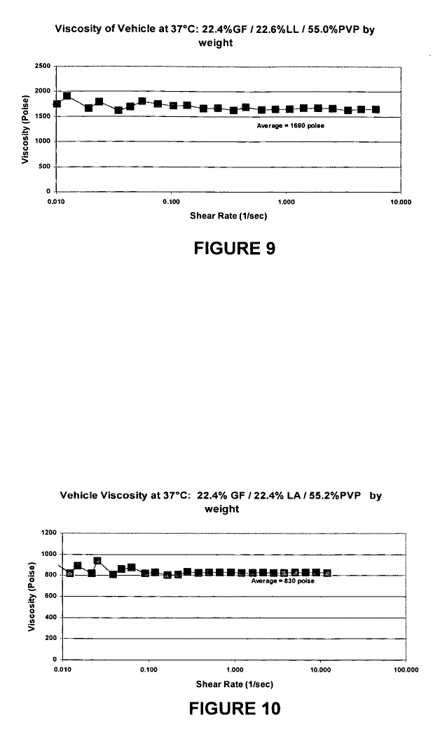

Viscosity of the suspending vehicle made up of 22.4 weight % GF, 22.6 weight % LL, and 55.0 weight % PVP was measured at various sheer rates. FIG. 9 demonstrates that viscosity is generally independent of shear rate. Average viscosity at 37° C. was 1690 poise.

The suspending vehicles were stored at 37° C., with no pharmaceutically active agents, for example, protein particles, present. An aqueous media, PBS, was incorporated into a sample of each suspending vehicle with a spatula at 10% by weight of the suspending vehicle. PBS was also incorporated into a different sample of each suspending vehicle at 20% by weight of the suspending vehicle.

Suspending vehicles were observed to discern whether a second phase formed upon contact with the aqueous media. When two phases were formed, the character of the aqueous (bottom) phase was observed.

### Example 6

GF was combined with another solvent to alter properties of vehicle, for example, soften a second phase formed when vehicle is exposed to water. Solvent combinations were screened to evaluate phase behaviors of different solvent ratios. Properties of the co-solvent vehicle in the presence of Phosphate Buffered Saline (PBS) at two levels (10% and 20% by weight) were evaluated.

Co-solvent mixtures were prepared at weight ratios of glycofurol to lauryl alcohol (LA) of 0:100, 10:90, 25:75, 50:50, and 75:25. For each ratio of GF to LA, the co-solvents were weighed and combined together in ajar. Dried polymer, PVP, was weighed and added to the jar at a solvent to polymer ratio of 45:55 for create a suspending vehicle. The suspending vehicle was mixed by hand with a spatula. After sufficient mixing, the jar was placed in an oven at 65° C. for approximately 1 hour to further dissolve any PVP. The jars were then placed in a vacuum oven and subjected to a vacuum for approximately 1 hour to remove any air entrapped in the suspending vehicle during mixing.

Viscosity of the suspending vehicle made up of 22.4 weight % GF, 22.4 weight % LA, and 55.5 weight % PVP was measured at various sheer rates. FIG. 10 demonstrates that viscosity is generally independent of shear rate. Average viscosity at 37° C. was 830 poise.

The suspending vehicles were stored at 37° C., with no pharmaceutically active agents, for example, protein particles, present. An aqueous media, PBS, was incorporated into a sample of each suspending vehicle with a spatula at 10% by weight of the suspending vehicle. PBS was also incorporated into a different sample of each suspending vehicle at 20% by weight of the suspending vehicle.

Suspending vehicles were observed to discern whether a second phase formed upon contact with the aqueous media. When two phases were formed, the character of the aqueous (bottom) phase was observed.

### Example 7

The suspending vehicles and aqueous phases of Examples 5 and 6 were compared. There was no qualitative difference discerned between aqueous phases resulting from vehicles having LA as the co-solvent as compared vehicles having LL as a co-solvent. Table 3 summarizes the phase behavior of the suspending vehicles of Examples 5 and 6, descriptions refer to, if any, the aqueous phases. As the glycofurol content increases, the bottom, aqueous phase softens. At 50:50 and 75:25 GF:LL or LA, one phase existed at 10% PBS, while lower glycofurol contents resulted in aqueous/organic phase separation. At higher glycofurol contents, one phase forms over a broader range of PBS content.

TABLE 3
Phase Behavior of GF/LL or LA/PVP Vehicles

| LL or LA Wt % | GF Wt % | 10% PBS | 20% PBS |
|---|---|---|---|
| 100 | 0 | Two Phases: | Two Phases: |
| | | Gritty and Hard | Gritty and Hard |
| 90 | 10 | Two Phases: Hard | Two Phases: Hard |
| 75 | 25 | Two Phases: | Two Phases: |
| | | Medium Hard | Medium Hard |
| 50 | 50 | One Phase | Two Phases: Soft |
| 25 | 75 | One Phase | Two Phases: |
| | | | Very Soft |

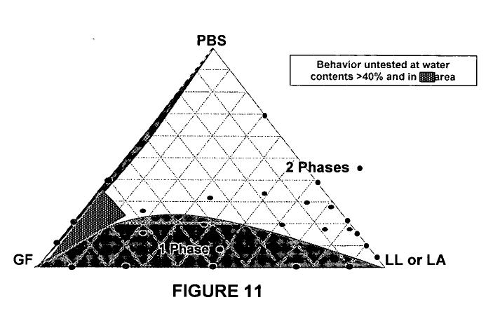

FIG. 11 shows a ternary phase diagram of suspending vehicles at varying compositions of GF and LL or LA and up to 40% added PBS. No difference was observed between LL and LA. The vehicle tested on the GF/PBS axis was 39:61 GF:PVP by weight. All other vehicles tested contained 45:55 SOLVENT:PVP. At higher glycofurol contents, one phase forms over a broader range of PBS content.

All publications, patents and patent applications cited herein are hereby incorporated by reference in their entirety. As used in this specification and the appended claims, the singular forms “a,” “an” and “the” include plural references unless the content clearly dictates otherwise.

Other aspects of the invention will be apparent from review of the present specification and claims and all such falling within the spirit of the invention are comprehended hereby.

What is claimed:

1. A suspending vehicle in a pump-driven dosage form comprising a hydrophobic solvent, a hydrophilic solvent, and a biocompatible polymer, wherein the suspending vehicle is substantially free of stiff gels upon contact with an aqueous medium.

2. The suspending vehicle of claim 1, wherein the polymer is a polyester, a pyrrolidone, an ester of unsaturated alcohols, an ether of unsaturated alcohols, a polyoxyethylenepolyoxypropylene block copolymer, or combinations thereof.

3. The suspending vehicle of claim 2 wherein the polymer comprises polyvinyl pyrolidone (PVP).

4. The suspending vehicle of claim 1 wherein a weight ratio of the hydrophobic solvent to the hydrophilic solvent is from about 50:50 to about 99:1.

5. The suspending vehicle of claim 4 wherein the weight ratio of the hydrophobic solvent to the hydrophilic solvent is from about 50:50 to about 90:10.

6. The suspending vehicle of claim 1 wherein a weight ratio of a combination of the hydrophilic solvent and the hydrophobic solvent to the polymer is from about 30:70 to about 70:30.

7. The suspending vehicle of claim 6 wherein the weight ratio of a combination of the hydrophilic solvent and the hydrophobic solvent to the polymer is from about 40:60 to about 55:45.

8. The suspending vehicle of claim 1 wherein the hydrophilic solvent comprises benzyl alcohol, triacetin, diacetin, tributyrin, triethyl citrate, tributyl citrate, acetyl triethyl citrate, acetyl tributyl citrate, triethyl phosphate, diethyl phthalate, diethyl tartrate, polybutene, silicone fluid, glycerine, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, 1-dodecylazacyclo-heptan-2-one, polysorbate 80, tetraglycol, or combinations thereof.

9. The suspending vehicle of claim 1 wherein the hydrophobic solvent comprises a carboxylic acid ester, a polyhydric alcohol, a polymer of a polyhydric alcohol, a fatty acid, an oil, propylene carbonate, an ester of a polyhydric alcohol, a triethylglyceride, or combinations thereof.

10. The suspending vehicle of claim 1 wherein the hydrophobic solvent comprises benzyl benzoate, lauryl alcohol, decyl alcohol, lauryl lactate, myristyl lactate, myristyl alcohol, decyl lactate, Ceraphyl® 31, ethyl oleate, ethyl hexyl lactate, a vegetable oil, vitamin E, oleic acid, a mineral oil, or combinations thereof.

11. The suspending vehicle of claim 1 wherein the suspending vehicle has a viscosity of from about 500 poise to about 70,000 poise at 37° C.

12. The suspending vehicle of claim 11 wherein the suspending vehicle has a viscosity of from about 5,000 poise to about 25,000 poise at 37° C.

13. A kit comprising the suspending vehicle of claim 1 and instructions for suspending or dispersing a pharmaceutically active agent therein to create a pharmaceutical suspension.

14. The kit of claim 13 further comprising a pump-driven dosage form and instructions for loading the dosage form with the pharmaceutical suspension.

15. A pharmaceutical suspension comprising a pharmaceutically active agent and a suspending vehicle, wherein the pharmaceutically active agent is suspended or dispersed in the suspending vehicle, wherein the suspending vehicle comprises a hydrophobic solvent, a hydrophilic solvent, and a biocompatible polymer and the suspending vehicle is substantially free of stiff gels upon contact with an aqueous medium.

16. The suspension of claim 15, wherein the polymer is a polyester, a pyrrolidone, an ester of unsaturated alcohols, an ether of unsaturated alcohols, a polyoxyethylenepolyoxypropylene block copolymer, or combinations thereof.

17. The suspension of claim 16 wherein the polymer comprises polyvinyl pyrolidone (PVP).

18. The suspension of claim 15 wherein the hydrophilic solvent comprises benzyl alcohol, triacetin, diacetin, tributyrin, triethyl citrate, tributyl citrate, acetyl triethyl citrate, acetyl tributyl citrate, triethyl phosphate, diethyl phthalate, diethyl tartrate, polybutene, silicone fluid, glycerine, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, 1-dodecylazacyclo-heptan-2-one, polysorbate 80, tetraglycol, or combinations thereof.

19. The suspension of claim 15 wherein the hydrophobic solvent comprises a carboxylic acid ester, a polyhydric alcohol, a polymer of a polyhydric alcohol, a fatty acid, an oil, propylene carbonate, an ester of a polyhydric alcohol, a triethylglyceride, or combinations thereof.

20. The suspension of claim 15 wherein the hydrophobic solvent comprises benzyl benzoate, lauryl alcohol, decyl alcohol, lauryl lactate, myristyl lactate, myristyl alcohol, decyl lactate, Ceraphyl® 31, ethyl oleate, ethyl hexyl lactate, a vegetable oil, vitamin E, oleic acid, a mineral oil, or combinations thereof.

21. The suspension of claim 15 wherein the pharmaceutically active agent comprises ω-interferon, α-interferon, β-interferon, γ-interferon, erythropoietin, granulocyte macrophage colony stimulating factor (GM-CSF), human growth hormone releasing hormone (huGHRH), insulin, desmopressin, infliximab, an antibody, an agent conjugated to a targeting ligand, bone morphogenic proteins, adrenocorticotropic hormone, angiotensin I, angiotensin II, atrial natriuretic peptide, bombesin, bradykinin, calcitonin, cerebellin, dynorphin N, alpha endorphin, beta endorphin, endothelin, enkephalin, epidermal growth factor, fertirelin, follicular gonadotropin releasing peptide, galanin, glucagon, glucagon-like peptide-1 (GLP-1), gonadorelin, gonadotropin, goserelin, growth hormone releasing peptide, histrelin, human growth hormone, insulin, leuprolide, LHRH, motilin, nafarerlin, neurotensin, oxytocin, relaxin, somatostatin, substance P, tumor necrosis factor, triptorelin, vasopressin, nerve growth factor, blood clotting factors, ribozymes, antisense oligonucleotide, or combinations thereof.

22. The suspension of claim 15 wherein the pharmaceutically active agent comprises risperidone, paliperidone, or combinations thereof.

23. A dosage form comprising:
a first wall that maintains its physical and chemical integrity during the life of the dosage form and is substantially impermeable to a pharmaceutical suspension;
a second wall that is partially permeable to an exterior fluid;
a compartment defined by the first wall and the second wall;
a pharmaceutical suspension that is positioned within the compartment and comprises a hydrophobic solvent, a hydrophilic solvent, a biocompatible polymer, and a pharmaceutically active agent, wherein the suspending vehicle is substantially free of stiff gels upon contact with an aqueous medium;
a pump in communication with the first wall, the second wall, and the compartment; and
an exit port in communication with the compartment.

24. The dosage form of claim 23 wherein the pump comprises an osmotic pump.

25. The dosage form of claim 23 wherein upon contact with an aqueous medium, the pharmaceutical suspension is flowable through the exit port under a force exerted by the pump under normal operating conditions.

26. The dosage form of claim 23 wherein the pharmaceutical suspension is substantially homogeneous for at least 3 months at 37° C.

27. A method comprising administering the dosage form of claim 23 to a mammal.

28. A kit comprising a dosage form of claim 23 and instructions for administering the dosage form.

29. A method comprising:
identifying a hydrophobic solvent;
identifying a hydrophilic solvent;
identifying a biocompatible polymer; and
mixing the hydrophilic solvent, the hydrophobic solvent, and the biocompatible polymer to create a suspending vehicle;
wherein the suspending vehicle is substantially free of stiff gels upon contact with an aqueous medium.

30. The method of claim 29 further comprising adding a pharmaceutically active agent to the suspending vehicle to create a pharmaceutical suspension.

31. The method of claim 30 further comprising adding the pharmaceutical suspension to a dosage form.

32. The method of claim 31 wherein the dosage form comprises a first wall that maintains its physical and chemical integrity during the life of the dosage form and is substantially impermeable to a pharmaceutical suspension; a second wall that is partially permeable to an exterior fluid; a compartment defined by the first wall and the second wall; a pump in communication with the first wall, the second wall, and the compartment; and an exit port in communication with the compartment; and wherein the pharmaceutical suspension is positioned within the compartment.
